(19) United States
(12) Patent Application Publication
(10) Pub. No.: US 2022/0301727 A1
(43) Pub. Date: Sep. 22, 2022

(54) SETTING UP VIDEO CALLS BETWEEN HEALTHCARE PROVIDERS AND PATIENTS

(71) Applicant: Doximity, Inc., San Francisco, CA (US)

(72) Inventors: Bruno Miranda, San Diego, CA (US); Jey Balachandran, San Francisco, CA (US); Rob Malko, Atlanta, GA (US); Jeremiah Konoske, Davis, CA (US); Joel Davis, San Francisco, CA (US)

(21) Appl. No.: 17/697,820

(22) Filed: Mar. 17, 2022

Related U.S. Application Data
(60) Provisional application No. 63/162,948, filed on Mar. 18, 2021.

(51) Int. Cl. G16H 80/00; H04L 9/32

(52) U.S. Cl. CPC G16H 80/00; H04L 9/3247

(57) ABSTRACT

A method for setting up a video call between a healthcare provider and a patient include receiving a request to initiate a video call between a healthcare provider device and a patient device, generating an online video call based on the request, generating a message comprising a link to the online video call, wherein the link allows the patient device to join the video call without using video call details, and providing the message comprising the link to the patient device.

### RELATED APPLICATIONS

This application claims priority from and the benefit of U.S. Provisional Patent Application No. 63/162,948 filed on Mar. 18, 2021, the entire contents of which are incorporated herein by reference in their entirety.

### TECHNICAL FIELD

This disclosure generally relates to video calls. In particular, this disclosure relates to setting up video calls between healthcare providers and patients.

### BACKGROUND

Healthcare providers (e.g., doctors, nurses, etc.) may provide various health related services and products to patients. Patients may often visit health care facilities (e.g., hospitals, clinics, etc.) to receive the health related services and products. For example, a patient may visit a clinic or a hospital for a checkup or to speak with a doctor about a particular medical/health issue.

### BRIEF DESCRIPTION OF THE DRAWINGS

Embodiments and implementations of the present disclosure will be understood more fully from the detailed description given below and from the accompanying drawings of various aspects and implementations of the disclosure, which, however, should not be taken to limit the disclosure to the specific embodiments or implementations, but are for explanation and understanding only.

FIG. 1 is a diagram illustrating an example network architecture in accordance with one or more embodiments of the disclosure.

FIG. 2 is a flow diagram of an example process of setting up a video call in accordance with one or more embodiments of the disclosure.

FIG. 3 is a diagram illustrating an example user interface in accordance with one or more embodiments of the disclosure.

FIG. 4 is a diagram illustrating another example user interface in accordance with one or more embodiments of the disclosure.

FIG. 5 is a flow diagram of an example process of setting up an online video call in accordance with one or more embodiments of the disclosure.

FIG. 6 is a flow diagram of another example of setting up an online video call for a patient in accordance with one or more embodiments of the disclosure.

FIG. 7 is a block diagram of an example apparatus that may perform one or more of the operations described herein, in accordance with some embodiments of the present disclosure.

### DETAILED DESCRIPTION

As discussed above, patients often visit healthcare professionals at physical locations to receive health related services and/or products. As healthcare continues to evolve, more efficient and/or convenient methods of providing health related services and/or products may be used. For example, rather than visiting a healthcare professional in person, it may be more convenient to have a video call (e.g., a video conference) between a healthcare professional and a user. However, a user may not be proficient in the use of computing devices and/or applications for joining or setting up video calls. For example, older patients may have more difficulty typing in an identifier for a video call and a password for the video call. Thus, an easier and/or more efficient method of joining or setting up a video call between a patient and a healthcare professional may be useful.

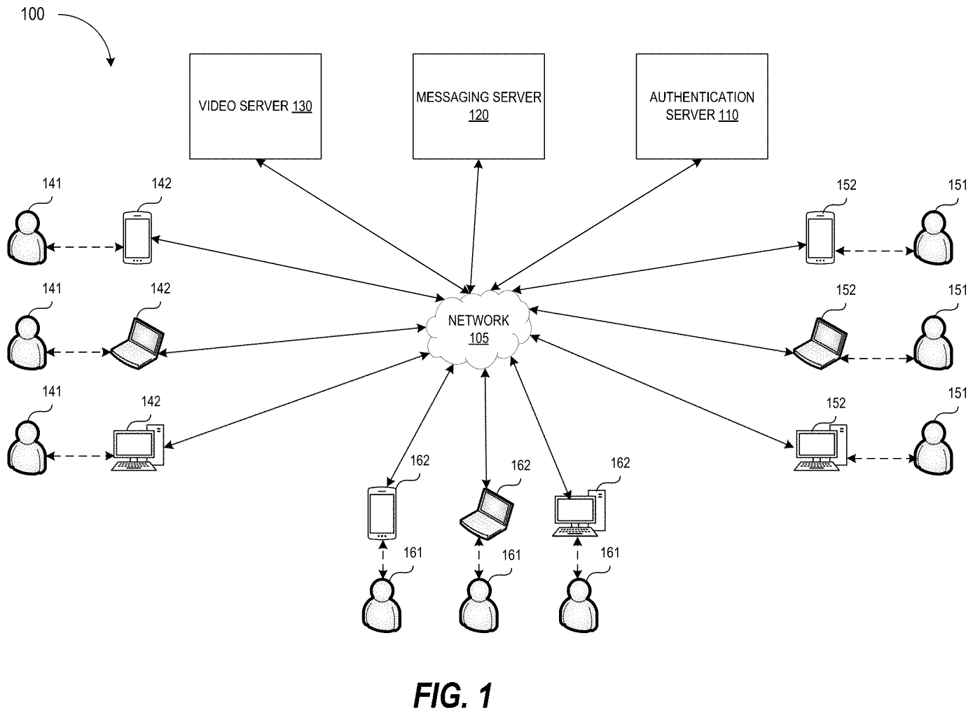

FIG. 1 is a diagram illustrating an example network architecture 100, in accordance with one or more embodiments of the disclosure. The network architecture 100 includes network 105, an authentication server 110, a messaging server 120, a video server 130, users 141, computing devices 142, healthcare providers 151, computing devices 152, service providers 161, and computing devices 162.

In one embodiment, the users 141 may be patients who use services and/or products provided by one or more of the healthcare providers 151 and service providers 161. Each user 141 may use a computing device 142 to communicate with one or more of a healthcare provider 151 and a service provider 161. Examples of computing devices 142 may include, but are not limited to, a smartphone, a tablet computer, a laptop computer, a desktop computer, etc.

In one embodiment, the healthcare providers 151 may be people who provide health related services and/or products to the user. Examples of healthcare providers 151 may include, but are not limited to, doctors, pharmacists, dentists, nurses, therapists, psychologists, technicians, surgeons, etc. Each healthcare provider 151 may use a computing device 152 (e.g., smartphone, tablet computer, etc.) to communicate with one or more of the users 141 and the service providers 161.

In one embodiment, a service provider 161 may provide additional or ancillary services to one or more of the users 141 and the healthcare providers 151. Examples of service providers 151 may include, but are not limited to, insurance providers, translators (e.g., language translators), billing specialists, etc. Each service provider 161 may use a computing device 162 (e.g., smartphone, tablet computer, etc.) to communicate with one or more of the users 141 and the healthcare providers 151.

As discussed above, a healthcare provider 151 (e.g., a doctor) may communicate with a user (e.g., a patient) via a video call (e.g., a video conference, a video meeting, etc.). The system architecture 100 may allow a healthcare provider 151 to provide a user 151 with a link that allows the user 151 to join or setup a video call with the healthcare provider 151. In one embodiment, the user 151 may be able to join or setup the video call without providing or using security credentials, such as a password, and/or without providing a meeting identifier.

In one embodiment, the authentication server 110 may authenticate one or more of the healthcare provider 151, a service provider 161, and a user 141. For example, a computing device 152 (used by the healthcare provider 151) may include an app (e.g., an application, a program, etc.) that allows the healthcare provider 151 to join or setup the video call with the user 141. The app may prompt the healthcare provider 151 for a user name, password, or other security credentials before allowing the user to join or setup the video call. The app may provide the security credentials to the authentication server 110 which may authenticate or verify the security credentials. After the security credentials of the healthcare provider 151 have been verified, the healthcare provider 151 may be allowed to setup or join the video call.

In one embodiment, the messaging server 120 may send or transmit the link to a computing device 142 of the user 141. For example, the healthcare provider 141 may setup a video call with the user via the app on the computing device 152. The app may communicate with the video server 130 and the video server 130 may communicate with the messaging server 120 to indicate that the healthcare provider 141 wants to setup a video call with the user 141. The video server 130 may generate a video call (e.g., a room, chat, etc.) on the video server 130. The messaging server 120 may transmit or send the link for the video call to the computing device 142 of the user. For example, the messaging server 120 may send a short message service (SMS) message to the computing device 142 of the user 141.

In one embodiment, the video server 130 may host the video call between the healthcare provider 151 and the user 141. For example, after the user selects, clicks, activates, etc., the link, the computing device 142 may communicate with the video server 130 to setup or join the video call. The video call may forward data (e.g., video data, video frames, audio data, audio frames, etc.) between the computing device 142 (of the user 141) and the computing device 152 (of the user 151). The video server 130 may also allow different people to join or leave a video call. For example, a first healthcare provider 151 may initiate the video call with the user 141. At a later point in time, the first healthcare provider 151 may add a second healthcare provider 151 to the video call and the first healthcare provider 151 may leave the video call. The video server 130 may forward data between the computing devices of the people who are added or removed from the video call.

In one embodiment, the link may allow the user 141 to join or setup the video call without providing or using security credentials, such as a password. For example, by selecting, clicking, activating, etc., the link, the computing device 142 of the user 141 may automatically setup or join a video call with the healthcare provider 151. The user 141 may not be prompted for security credentials to join or setup the video call. For example, the link may include credentials and/or other information necessary to securely join the video call.

In one embodiment, the link may include various portions or pieces of data. For example, the link may include one or more of a location of a server (e.g., the video server 130), one or more identifiers, a time period, and a signature. The link may allow the user 141 to join the video call without providing security credentials (e.g., without entering a password) and/or without providing an identifier for the meeting (e.g., a meeting ID, a meeting name, etc.). This may allow the user 141 to join the video call more quickly and/or easily. The different portions of pieces of data link in the link are discussed in more detail below. Although some examples and embodiments are described herein with respect to a healthcare provider, a service provider, or a user (e.g., a patient) it should be noted that embodiments may be used in any context in which a video call is initiated for others to join. Furthermore, any actions described herein as being performed by a healthcare provider, a service provider, or a user may be performed by a computing device of the healthcare provider, service provider, or user.

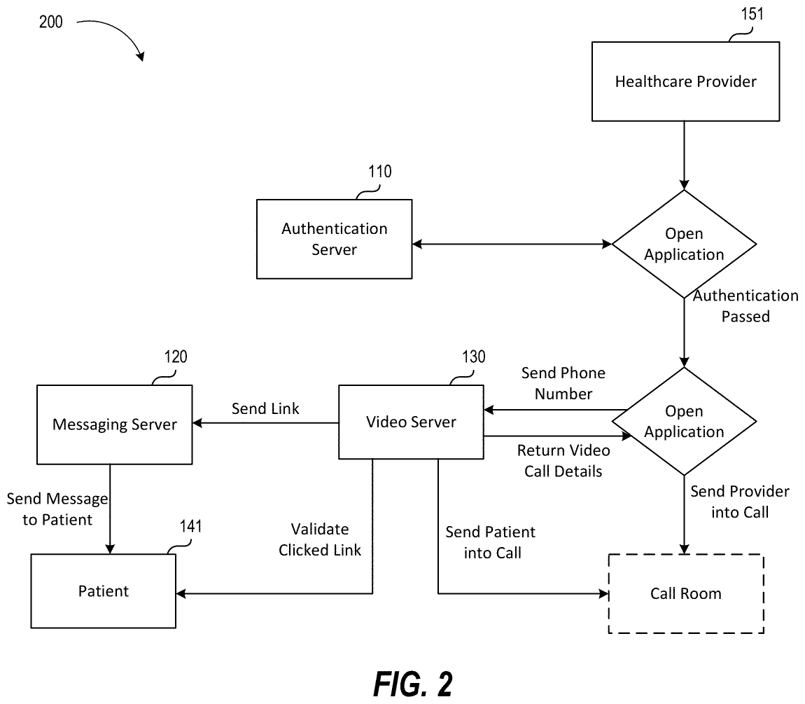

FIG. 2 is a flow diagram of a process 200 of setting up a video call in accordance with one or more embodiments of the present disclosure. Process 200 may be performed by processing logic that may comprise hardware (e.g., circuitry, dedicated logic, programmable logic, a processor, a processing device, a central processing unit (CPU), a system-on-chip (SoC), etc.), software (e.g., instructions running/executing on a processing device), firmware (e.g., microcode), or a combination thereof. In some embodiments, the process 200 may be performed by various computing devices, such as an authentication server, a messaging server, a video server, etc. The actions of the user 141 may be performed by a computing device of the user 141 (e.g., computing device 142 illustrated in FIG. 1) and the actions of the healthcare provider 151 may be performed by a computing device of the healthcare provider 151 (e.g., computing device 152 illustrated in FIG. 1).

The process 200 begins at block 205, wherein the process a healthcare provider 151 (e.g., a doctor) may use, open, etc., an app (e.g., an application, software, etc.) to setup or initiate a video call with the user 141 (e.g., a patient). The user may be prompted for security credentials which may be provided to the authentication server 110 for verification or authentication. If the security credentials are verified, the app may initiate a video call and may communicate with the video server 130 to setup the video call, which the user may join at a later time. The healthcare provider 151 may also provide a phone number, email address, name, etc., of the user 141 so that the messaging server 210 is able to determine which user to send the message and/or link to.

The video server 130 may also communicate with the messaging server 120 to indicate that the healthcare professional 151 wants to setup a video call with the user 141. The messaging server 120 may transmit message (e.g., an SMS message, a MMS message, a chat message, a text message, etc.) to the user 141 to indicate that the healthcare professional 151 wants to setup a video call with the user 141. The message may include a link that allows the user 141 to join the video call. The link may allow the user 141 to join the video call without providing security credentials (e.g., without entering a password). This may allow the user 141 to join the video call more quickly and/or easily. The link is discussed in more detail below.

When the user 141 activates, clicks, selects, etc., the link, the user may automatically join the video call initiated by the healthcare professional 151. The user 141 may be connected to the video server 130 so that the video server 130 can allow the user 141 to join the video call with the healthcare provider 151. For example, the video server 130 may place the healthcare provider 151 and the user 141 in a room (e.g., a call room, a video meeting room, a virtual room, etc.).

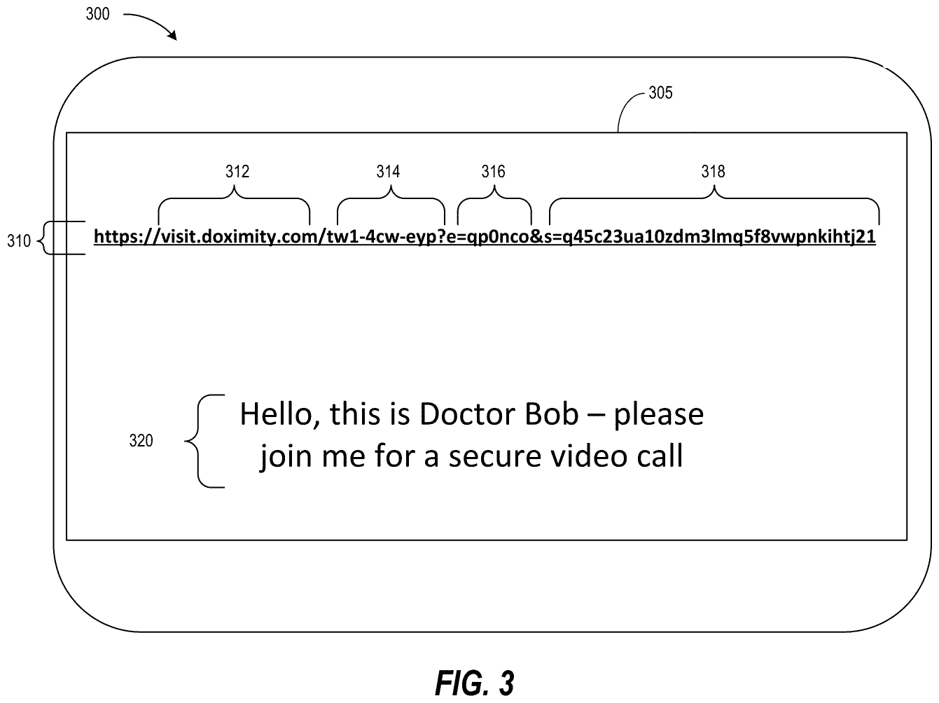

FIG. 3 is a diagram illustrating an example user interface 300 in accordance with one or more embodiments of the disclosure. The user interface 300 may display a message 305 received from a messaging server (e.g., messaging server 120 illustrated in FIG. 1). The message 305 may indicate to a user (e.g., a patient) that a healthcare provider (e.g., a doctor, nurse, pharmacist, etc.) wants to initiate a video call with the user. The messages 305 includes a link 310 and additional text 320. The additional text 320 may convey additional information to the user, such as which healthcare provider is initiating the video call (e.g., a name of the healthcare provider), when the video call is scheduled, the purpose of the video call (e.g., a checkup, a follow-up consultation, etc.). In one embodiment, the link 310 may be a uniform resources locator (URL). Although the present disclosure refers to a URL, other types of resource locators, identifiers, resource identifiers, etc., may be use in other embodiments.

In one embodiment, the “https://” portion of the link 310 may indicate the communication protocol that may be used to setup or join the video call. For example, the “https://” portion may indicate that the Hypertext Transfer Protocol (HTTP) along with transport layer security (TLS) may be used to setup or join the video call.

In one embodiment, the “visit.doximity.com” portion of the link 310 may indicate a location 312 for the video server that may host the video call between the user and the healthcare professional. Different locations (e.g., different domain names) may be used to identify and locate different video servers. In some examples, the link may also include an indication of a path to route video data for the video call.

In one embodiment, the portion “tw1-4cw-eyp” of the link 310 may be an identifier 314 for the video call. The identifier 314 may be a unique identifier of the video call. For example, the identifier 314 may be unique within the domain name and/or video server. The identifier 314 may not be case sensitive in some embodiments. The format of the identifier 314 may vary in different embodiments. For example, any communication alphanumeric characters may be used in the identifier. The “?” portion of the link 310 may be a separator between different portions of the link 310.

In one embodiment, the “E=qp0nco” portion of the of the link 310 may indicate or represent, a time or time period 316 for the video call. For example, the “E=qp0nco” portion may indicate a start time for the video call (e.g., a time when video call should start). In another example, the “E=qp0nco” portion may indicate a time frame in which the video call should occur (e.g., may indicate a start time and an end time for the video call). In a further example, the “E=qp0nco” portion may indicate an expiration time (e.g., a latest time) within which the user is able to join or setup the video call.

The “S=q45c23ua10zdm31mq5fvwpnkihtj21” portion of the of the link 310 may be a signature 318 to help increase the security of the video call. The “S=q45c23ua10zdm31mq5f8vwpnkihtj21” portion may prevent a malicious user from modifying the link 310. If the link 310 has been modified by the malicious user, the video server will reject the request to join or setup the video call if the signature does not match the signature obtained by the video server. The “S=q45c23ua10zdm31mq5f8vwpnkihtj21” portion may also prevent unauthorized users from joining the video call. For example, the “S=q45c23ua10zdm31mq5f8vwpnkihtj21” portion may be generated using various encryption and/or hashing algorithms. In one embodiment, the “S=q45c23ua10zdm31mq5fvwpnkihtj21” portion may be generated by hashing the previous portions of the link 310 using a hash-based message authentication code secure hash algorithm (HMAC-SHA). This may prevent a malicious user from guessing the signature in an attempt to join the video call.

As discussed above, the link 310 allows a user (e.g., a patient) to join or setup a video call with a healthcare provider more easily. For example, when the user clicks, selects, etc., the link 310, the video call may be automatically initiated. The user may not need to provide security credentials (e.g., a password) to join the video call. This allows a user to join the video call with fewer steps.

In other embodiments, the message 305 may be provided to other people, such as service providers and/or other healthcare professional. For example, the message 305 may be forwarded to a second healthcare provider (e.g., a specialist), a service provider (e.g., a Spanish language translator), etc.

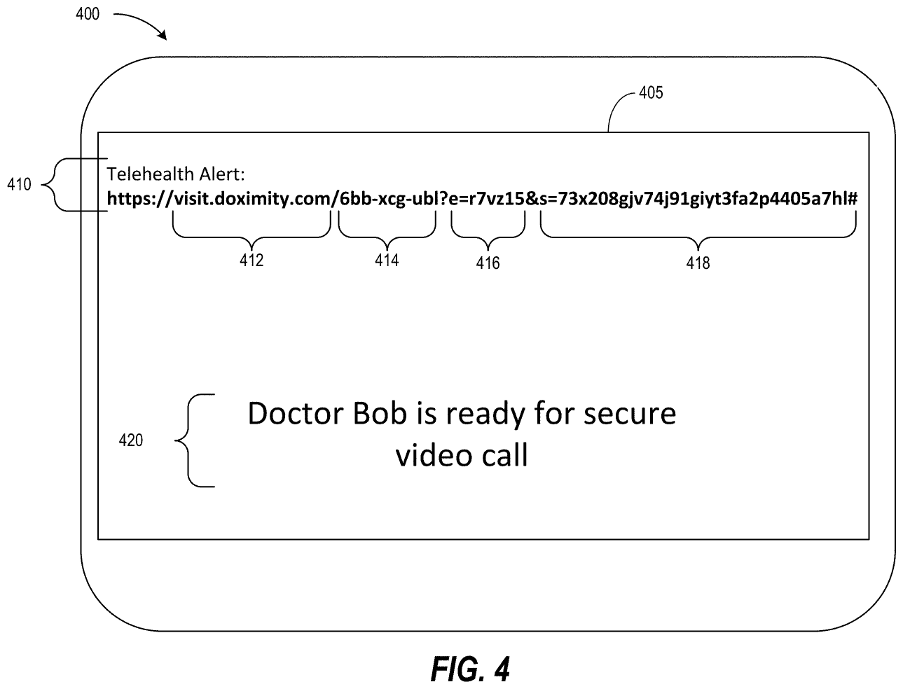

FIG. 4 depicts a diagram illustrating an example user interface 400 in accordance with one or more embodiments of the disclosure. Similar to user interface 300 of FIG. 3, user interface 400 may display a message 405 received from a messaging server (e.g., messaging server 120 illustrated in FIG. 1). The message 405 may indicate to a user (e.g., a patient) that a healthcare provider (e.g., a doctor, nurse, pharmacist, etc.) wants to initiate a video call with the user. The message 405 includes a link 410 and additional text 420. The additional text 420 may convey additional information to the user, such as which healthcare provider is initiating the video call (e.g., a name of the healthcare provider), when the video call is scheduled, the purpose of the video call (e.g., a checkup, a follow-up consultation, etc.). In one embodiment, the link 410 may be a uniform resources locator (URL). Although the present disclosure refers to a URL, other types of resource locators, identifiers, resource identifiers, etc., may be use in other embodiments.

The link 410 provided in the message 405 may include a location 412 of the video call, an identifier of the video call 414, a time 416 associated with the video call, and a signature 418. The location 412 of the video call (e.g., “visit.doximity.com”) may be a location of a location of a server, website, application, or the like. The identifier 414 of the video call (e.g., “6bb-xcg-ubl”) may direct the request to a specific call being executed on the server identified by the location 412. The time 416 component of the link (e.g., “e=r7vz15”) may identify a start time, duration, expiration time, and/or any other time or scheduling related specifics of the video call. The signature 418 string (e.g., “s=73x208gjv74j91giyt3fa2p4405a7hl#”) may be a unique alpha-numerical string required as a key to access the video call. For example, the signature 418 may be a hash or encryption based on the other components of the link. In some examples, the signature 418 may be a randomly generated string. In some examples, the signature 418 may allow only users with the link to access the video chat.

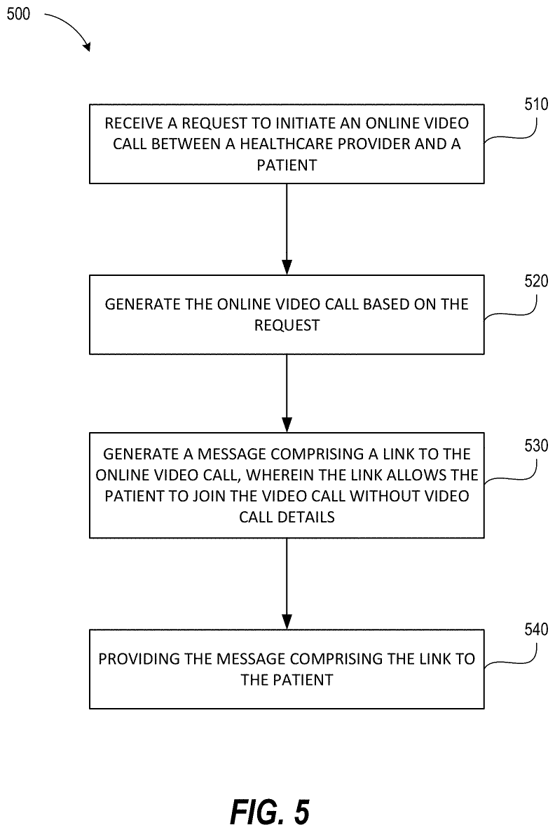

FIG. 5 is a flow diagram of a process 500 of setting up an online video call in accordance with one or more embodiments of the present disclosure. Process 500 may be performed by processing logic that may comprise hardware (e.g., circuitry, dedicated logic, programmable logic, a processor, a processing device, a central processing unit (CPU), a system-on-chip (SoC), etc.), software (e.g., instructions running/executing on a processing device), firmware (e.g., microcode), or a combination thereof. In some embodiments, the process 500 may be performed by various computing devices, such as an authentication server, a messaging server, a video server, etc. The actions of the user 141 may be performed by a computing device of the user 141 (e.g., computing device 142 illustrated in FIG. 1) and the actions of the healthcare provider 151 may be performed by a computing device of the healthcare provider 151 (e.g., computing device 152 illustrated in FIG. 1).

Process 500 begins at block 510, where processing logic receives a request to initiate an online video call between a healthcare provider and a patient. For example, the healthcare provider may access an interface of an application for initiating a video call. The application may be a local application, a web application, or any other software application. The healthcare provider may enter information for the patient that is to join the video call. For example, the healthcare provider may enter a patient name, phone number, patient number, or other identifying information as well as a time at which the video call is to take place. In some examples, the healthcare provider may also provide credentials. The processing logic (e.g., an authentication server) may then authenticate the request from the healthcare provider based on the provided credentials.

At block 520, the processing logic generates the online video call based on the request. For example, the processing logic may generate a video call “room” on a video call server. The video call room may broker (e.g., receive and forward) video and audio information between devices of the healthcare provider, the patient, and any additional participants of the video call.

At block 530, the processing logic generates a message comprising a link to the online video call, wherein the link allows the patient to join the video call without video call details. The link may include an identifier of the video call server hosting the online video call and an identifier of the video call (e.g., the video call room on the server). The link may also include a time period for the online video call (e.g., start time, duration, end time, expiration time, etc.) and a unique signature for the call. For example, the signature may be an alpha-numeric string generated based on a hash or encryption algorithm. In some examples, the signature may be a hash of the other components (e.g., the video server identifier, video call identifier, etc.). Accordingly, the link may include information to allow the patient, or any other participants with the link, to join the video call directly from the message by selecting, following, or opening the link.

At block 540, the processing logic provides the message comprising the link to the patient. The patient may then select the link and be directly sent into the video call. For example, the patient may not have to enter any credentials or meeting information beyond clicking the provided link. In some examples, the link can also be forwarded to additional users, such as additional healthcare professionals, service providers, etc. allowing the additional users to also join the online video call by selecting the link. In some examples, the healthcare provider may pass the patient off another healthcare provider that has received and selected the link.

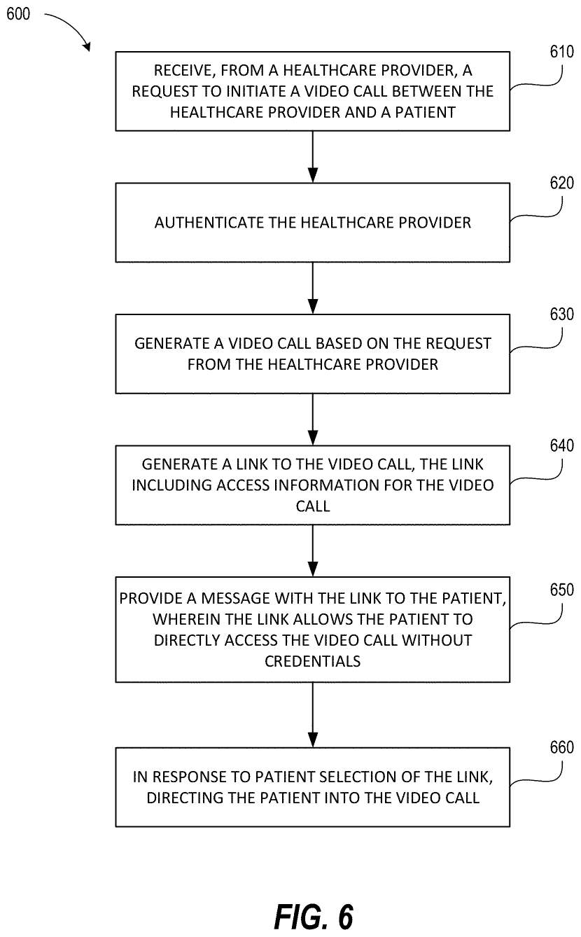

FIG. 6 is a flow diagram of a process 600 of setting up an online video call in accordance with one or more embodiments of the present disclosure. Process 600 may be performed by processing logic that may comprise hardware (e.g., circuitry, dedicated logic, programmable logic, a processor, a processing device, a central processing unit (CPU), a system-on-chip (SoC), etc.), software (e.g., instructions running/executing on a processing device), firmware (e.g., microcode), or a combination thereof. In some embodiments, the process 600 may be performed by various computing devices, such as an authentication server, a messaging server, a video server, etc. The actions of the user 141 may be performed by a computing device of the user 141 (e.g., computing device 142 illustrated in FIG. 1) and the actions of the healthcare provider 151 may be performed by a computing device of the healthcare provider 151 (e.g., computing device 152 illustrated in FIG. 1).

Process 600 begins at block 610, where processing logic receives, from a healthcare provider, a request to initiate a video call between the healthcare provider and a patient. At block 620, the processing logic authenticates the healthcare provider. At block 630, the processing logic generates a video call based on the request from the healthcare provider. At block 640, the processing logic generates a link to the video call, the link including access information for the video call. At block 650, the processing logic provides a message with the link to the patient, wherein the link allows the patient to directly access the video call without credentials. At block 660, in response to patient selection of the link, the processing logic directs the patient into the video call.

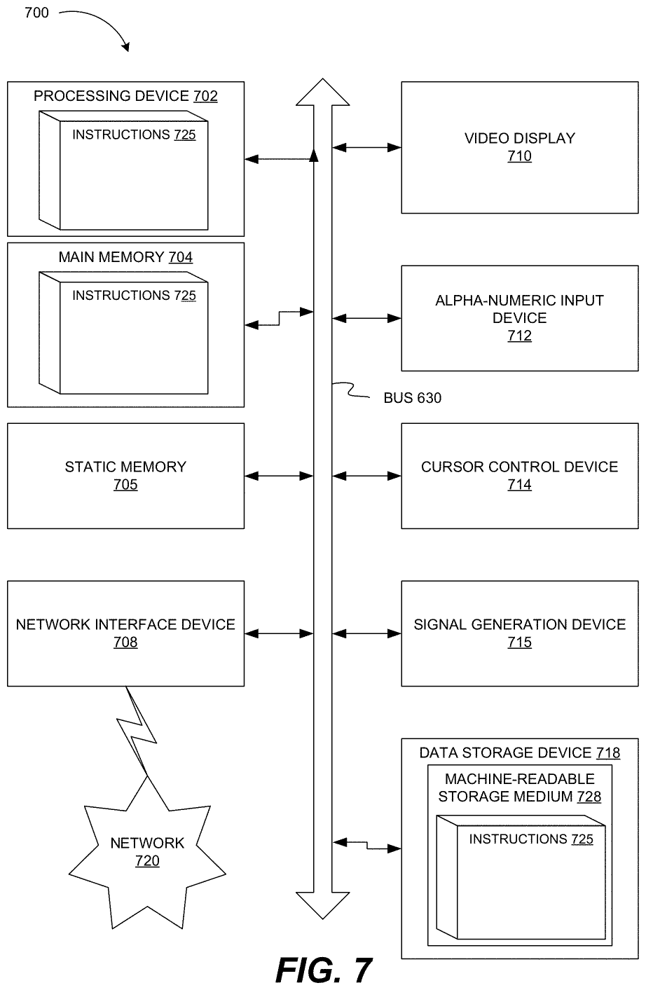

FIG. 7 is a block diagram of an example computing device 700 that may perform one or more of the operations described herein, in accordance with some embodiments. Computing device 700 may be connected to other computing devices in a LAN, an intranet, an extranet, and/or the Internet. The computing device may operate in the capacity of a server machine in the client-server network environment or in the capacity of a client in a peer-to-peer network environment. The computing device may be provided by a personal computer (PC), a set-top box (STB), a server, a network router, switch or bridge, or any machine capable of executing a set of instructions (sequential or otherwise) that specify actions to be taken by that machine. Further, while only a single computing device is illustrated, the term “computing device” shall also be taken to include any collection of computing devices that individually or jointly execute a set (or multiple sets) of instructions to perform the methods discussed herein.

The example computing device 700 may include a processing device (e.g., a general purpose processor, a PLD, etc.) 702, a main memory 704 (e.g., synchronous dynamic random access memory (DRAM), read-only memory (ROM)), a static memory 706 (e.g., flash memory and a data storage device 718), which may communicate with each other via a bus 730.

Processing device 702 may be provided by one or more general-purpose processing devices such as a microprocessor, central processing unit, or the like. In an illustrative example, processing device 702 may comprise a complex instruction set computing (CISC) microprocessor, reduced instruction set computing (RISC) microprocessor, very long instruction word (VLIW) microprocessor, or a processor implementing other instruction sets or processors implementing a combination of instruction sets. Processing device 702 may also comprise one or more special-purpose processing devices such as an application specific integrated circuit (ASIC), a field programmable gate array (FPGA), a digital signal processor (DSP), network processor, or the like. The processing device 702 may be configured to execute the operations described herein, in accordance with one or more aspects of the present disclosure, for performing the operations and steps discussed herein.

Computing device 700 may further include a network interface device 708 which may communicate with a network 720. The computing device 700 also may include a video display unit 710 (e.g., a liquid crystal display (LCD) or a cathode ray tube (CRT)), an alphanumeric input device 712 (e.g., a keyboard), a cursor control device 714 (e.g., a mouse) and an acoustic signal generation device 716 (e.g., a speaker). In one embodiment, video display unit 710, alphanumeric input device 712, and cursor control device 714 may be combined into a single component or device (e.g., an LCD touch screen).

Data storage device 718 may include a computer-readable storage medium 728 on which may be stored one or more sets of instructions 725 that may include instructions for carrying out the operations described herein, in accordance with one or more aspects of the present disclosure. Instructions 725 may also reside, completely or at least partially, within main memory 704 and/or within processing device 702 during execution thereof by computing device 700, main memory 704 and processing device 702 also constituting computer-readable media. The instructions 725 may further be transmitted or received over a network 720 via network interface device 708.

While computer-readable storage medium 728 is shown in an illustrative example to be a single medium, the term “computer-readable storage medium” should be taken to include a single medium or multiple media (e.g., a centralized or distributed database and/or associated caches and servers) that store the one or more sets of instructions. The term “computer-readable storage medium” shall also be taken to include any medium that is capable of storing, encoding or carrying a set of instructions for execution by the machine and that cause the machine to perform the methods described herein. The term “computer-readable storage medium” shall accordingly be taken to include, but not be limited to, solid-state memories, optical media and magnetic media.

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the embodiments and its practical applications, to thereby enable others skilled in the art to best utilize the embodiments and various modifications as may be suited to the particular use contemplated. Accordingly, the present embodiments are to be considered as illustrative and not restrictive, and the invention is not to be limited to the details given herein, but may be modified within the scope and equivalents of the appended claims.

What is claimed is:

1. A method comprising:
receiving a request to initiate a video call between a healthcare provider device and a patient device;
generating, by a processing device, an online video call based on the request;
generating, by the processing device, a message comprising a link to the online video call, wherein the link allows the patient device to join the video call without using video call details;
providing, by the processing device, the message comprising the link to the patient device.

2. The method of claim 1, wherein the link comprises:
an identifier for a video server associated with the online video call;
an identifier for the online video call;
a time period for the online video call; and
a signature.

3. The method of claim 2, wherein the signature is a unique string generated based on a hash or encryption applied to the identifier of the video server and the identifier for the online video call.

4. The method of claim 1, further comprising:
authenticating the request to initiate the video call based on credentials received with the request.

5. The method of claim 1, wherein the link allows the patient device to join the video call directly from the message.

6. The method of claim 5, wherein the link allows the patient device to join the video call without a password or an identifier of the online video call.

7. The method of claim 1, further comprising:
forwarding the link to one or more additional user devices; and
allowing the one or more additional user devices to join the online video call by selecting the link.

8. A system comprising:
a memory; and
a processing device, operatively coupled to the memory, to:
receive a request to initiate a video call between a healthcare provider device and a patient device;
generate an online video call based on the request;
generate a message comprising a link to the online video call, wherein the link allows the patient device to join the video call without using video call details; and
provide the message comprising the link to the patient device.

9. The system of claim 8, wherein the link comprises:
an identifier for a video server associated with the online video call;
an identifier for the online video call;
a time period for the online video call; and
a signature.

10. The system of claim 9, wherein the signature is a unique string generated based on a hash or encryption applied to the identifier of the video server and the identifier for the online video call.

11. The system of claim 8, wherein the processing device is to:
authenticate the request to initiate the video call based on credentials received with the request.

12. The system of claim 8, wherein the link allows the patient device to join the video call directly from the message.

13. The system of claim 12, wherein the link allows the patient device to join the video call without a password or an identifier of the online video call.

14. The system of claim 8, wherein the processing device is to:
forward the link to one or more additional user devices; and
allow the one or more additional user devices to join the online video call by selecting the link.

15. A non-transitory computer readable storage medium including instructions stored therein, that when executed by a processing device, cause the processing device to:
receive a request to initiate a video call between a healthcare provider device and a patient device;
generate, by the processing device, an online video call based on the request;
generate, by the processing device, a message comprising a link to the online video call, wherein the link allows the patient device to join the video call without using video call details; and
provide, by the processing device, the message comprising the link to the patient device.

16. The system of claim 15, wherein the link comprises:
an identifier for a video server associated with the online video call;
an identifier for the online video call;
a time period for the online video call; and
a signature.

17. The system of claim 16, wherein the signature is a unique string generated based on a hash or encryption applied to the identifier of the video server and the identifier for the online video call.

18. The system of claim 15, wherein the processing device is to:
authenticate the request to initiate the video call based on credentials received with the request.

19. The system of claim 15, wherein the link allows the patient device to join the video call directly from the message.

20. The system of claim 19, wherein the link allows the patient device to join the video call without a password or an identifier of the online video call.
